(19) United States
(12) Patent Application Publication
(10) Pub. No.: US 2026/0262862 A1
(43) Pub. Date: Sep. 10, 2026

(54) WEARING ASSISTANCE TOOL AND WEARING ASSISTANCE KIT FOR ELASTIC STOCKING, AND WEARING PREPARATION METHOD AND WEARING METHOD FOR ELASTIC STOCKING

(71) Applicant: NATIONAL UNIVERSITY CORPORATION HAMAMATSU UNIVERSITY SCHOOL OF MEDICINE, Hamamatsu-shi, Shizuoka (JP)

(72) Inventors: Masaki SANO, Shizuoka (JP); Yurina YOKOYAMA, Shizuoka (JP); Toshiaki SUZUKI, Shizuoka (JP)

(21) Appl. No.: 19/166,302

(22) PCT Filed: Mar. 22, 2025
(86) PCT No.: PCT/JP2024/011194

§ 371 Date: Sep. 17, 2025

(30) Foreign Application Priority Data
Mar. 24, 2023 (JP) 2023-048485

(51) Int. Cl. A47G 25/90; A61F 13/08

(52) U.S. Cl. CPC A47G 25/905; A61F 13/08

(57) ABSTRACT

A donning assistance tool includes a plurality of ring-forming members and a connecting body that is formed of a stretchable and contractible material and that connects the ring-forming members. The size of the ring-forming member ranges from 10 mm to 30 mm. The ring-forming member has a through hole into which the connecting body is inserted. Regarding the size of a ring body in a non-stretched state formed by inserting the connecting body into the through hole, the diameter ranges from 40 mm to 90 mm in placing the ring body such that a virtual line inscribed in the ring-forming members constituting the ring body is a circle.

### TECHNICAL FIELD

The disclosure of the present application relates to a donning assistance tool and a donning assistance kit for an elastic garment, and a donning preparation method and a donning method for an elastic garment.

For edema of the extremities caused by abnormalities of the veins and lymphatic vessels, physical therapy using elastic garments (elastic stockings, ES) is effective. The elastic garment is also effective for disuse edema in bedridden persons and wheelchair users, effective in the prevention of economy class syndrome (phlebothrombosis), and the like. The market is expanding to the nursing care industry as well as the general public. The elastic garments are in various types, including different lengths, compression pressures, and the like, and are used separately for specific applications. However, most elderly persons have weak muscles and are sometimes unable to don and doff elastic garments. For this reason, a donning assistance tool for an elastic garment is known. As for the donning assistance tool, there are three known types: (1) a pull-out type, (2) a pull-up type, and (3) a roll-up type.

(1) As an example of the pull-out type, Magnide (trade name) of Arion Group, Netherlands, is known. FIG. 1 is a photograph showing the example use of Magnide. In donning the elastic garment, the folded Magnide is unfolded (a, b), the Magnide is donned onto the toe (c), and then the elastic garment is donned onto the Magnide (d). After the elastic garment is pulled up to above the heel, the Magnide is pulled out (see an arrow in e). As other examples of the pull-out type, a method described in Patent Literature 1 is also known.

(2) As an example of the pull-up type, Stocking Donner (trade name) of BSN Medical GmbH, Germany, is known. FIG. 2 is a photograph showing the example use of Stocking Donner. In donning the elastic garment, to the circular part of Stocking Donner (a), the elastic garment is placed (b), the elastic garment is lowered inside the circular part to fold outward at the circular part, and the elastic garment is put on the circular part (c), the toe is inserted into the circular part to which the elastic garment is put (d), and the entire elastic garment is pulled up (see an arrow in e).

(3) As an example of the roll-up type, Doff N′Donner (trade name) of SIGVARIS GROUP is known. FIG. 3 is a photograph showing the example use of Doff N′Donner. In donning the elastic garment, Doff N′Donner is used together with a cone, which is an accessory (a); the elastic garment is placed over the cone, the main part of Doff N′Donner is placed over the elastic garment, the main part is slid up and down to roll the elastic garment on the main part (b, c); the toe is inserted into the main part around which the elastic garment rolls (d), the elastic garment is pulled up as sliding over the skin (see an arrow in e); and after the elastic garment is donned over the leg, the main part of Doff N′Donner remaining on the leg is detached (f).

### CITATION LIST

### Patent Literature

PATENT LITERATURE 1: JP-A-2014-217417

### SUMMARY OF INVENTION

### Problems to be Solved by Invention

However, Magnide, shown in FIG. 1, has a problem of not being suited for donning an above-the-knee elastic garment. Moreover, Magnide also has a problem of being unusable to elderly persons with fragile skin and patients with skin ulcers, as Magnide irritates the skin. Moreover, the previously existing donning assistance tools are sold as dedicated tools. As is apparent from FIG. 1 to FIG. 3, the previously existing donning assistance tools are relatively large, and have a problem of being inconvenient for portability.

The disclosure of the present application is made to solve the problems. The present inventors have diligently investigated and found a donning assistance tool capable of solving the problems. That is, a ring body formed using (1) a plurality of ring-forming members in a predetermined size and (2) a connecting body made of a stretchable and contractible material and connecting ring-forming members, and (3) inserting a connecting body into the ring-forming members, is formed in a predetermined size to reduce the size of the donning assistance tool. (4) Since the size of the ring body is variable, a commercially available plastic bottle and the like can be used as a tubular product that supports an elastic garment, and it is unnecessary to carry a tubular product.

That is, it is an object of the disclosure of the present application to provide a donning assistance tool and a donning assistance kit for an elastic garment, and a donning preparation method and a donning method for an elastic garment, all of which improve portability convenience.

### Solution to Problems

The disclosure of the present application relates to a donning assistance tool and a donning assistance kit for an elastic garment, and a donning preparation method and a donning method for an elastic garment shown below.

(1) A donning assistance tool for an elastic garment, the donning assistance tool including: a plurality of ring-forming members; and a connecting body that is formed of a stretchable and contractible material and that connects the ring-forming members, in which the ring-forming member has a size ranging from 10 mm to 30 mm, and a through hole into which the connecting body is inserted, and regarding a size in a non-stretched state of a ring body formed by inserting the connecting body into the through hole, a diameter in placing the ring body such that a virtual line inscribed in the ring-forming members constituting the ring body ranges from 40 mm to 130 mm.

(2) The donning assistance tool according to (1), in which the diameter ranges from 40 mm to 70 mm.

(3) The donning assistance tool according to (1), in which the diameter ranges from 40 mm to 90 mm.

(4) The donning assistance tool according to (1), in which the connecting body is stretchable three to four times based on a length of the connecting body in a non-stretched state of the ring body.

(5) The donning assistance tool according to (1), in which the connecting body is in a seamless ring shape, the ring-forming member has a cut portion reaching from a through hole to an outer edge of the ring-forming member, and the connecting body is insertable into the through hole via the cut portion.

(6) The donning assistance tool according to (5), in which in the ring-forming member and the connecting body, when the connecting body is stretched, the ring-forming member is rotatable and movable with respect to the connecting body, and compared to when stretched, when the connecting body is not stretched, the ring-forming member and the connecting body are formed such that rotation and moving properties of the ring-forming member with respect to the connecting body are reduced.

(7) A donning assistance kit for an elastic garment, the donning assistance kit including: the donning assistance tool according to any one of (1) to (6); and a friction adjustment sheet, in which friction of the friction adjustment sheet moved in a state in which the elastic garment is contacted is varied between a first direction and a second direction that is a direction opposite to the first direction.

(8) A donning assistance kit for an elastic garment, the donning assistance kit including: the donning assistance tool according to any one of (1) to (6); and a cup having an outer diameter of 69 mm or an outer diameter of 90 mm.

(9) A donning assistance kit for an elastic garment, the donning assistance kit including: the donning assistance tool according to any one of (1) to (6); a friction in adjustment sheet; and a cup having an outer diameter of 69 mm or an outer diameter of 90 mm, in which friction of the friction adjustment sheet moved in a state in which the elastic garment is contacted is varied between a first direction and a second direction that is a direction opposite to the first direction.

(10) A donning assistance kit for an elastic garment, the donning assistance kit including: the donning assistance tool according to any one of (1) to (6); and a tubular product having an outer diameter ranging from 36 mm to 143 mm, in which an outer diameter of the tubular product is equal to a diameter of the ring body in a non-stretched state, or the outer diameter of the tubular product is within a range ±10% a diameter of the ring body in a non-stretched state.

(11) A donning preparation method for an elastic garment, in which the donning preparation method is performed at least using the donning assistance tool according to any one of (1) to (6), and a tubular product, and the donning preparation method includes an elastic garment end portion attaching process in which an end portion of the elastic garment is attached to the tubular product while sliding from a first end portion side to a second end portion side of the tubular product, an elastic garment rolling up process in which the donning assistance tool is placed on the end portion of the elastic garment attached to the tubular product, the donning assistance tool is moved while rotating from the second end portion side to the first end portion side of the tubular product along the tubular product, and the elastic garment is rolled up around the donning assistance tool, an elastic garment re-attaching process in which the donning assistance tool around which the elastic garment is rolled up is slid from the first end portion side to the second end portion side, and the elastic garment is again attached to the tubular product, and an attached elastic garment re-rolling up process in which the donning assistance tool is moved while rotating from the second end portion side to the first end portion side of the tubular product along the tubular product, and the elastic garment again attached to the donning assistance tool is rolled up.

(12) The donning preparation method according (11), in which after the attached elastic garment re-rolling up process, the elastic garment re-attaching process and the attached elastic garment re-rolling up process are repeatedly performed.

(13) The donning preparation method according to (11), in which the elastic garment rolling up process and/or the attached elastic garment re-rolling up process are performed in a state in which the tubular product is inverted such that a direction from the second end portion side to the first end portion side is a gravity direction.

(14) The donning preparation method according to (11), further including a different friction adjustment sheet in which friction of the elastic garment in moving in a contacted state is different in a first direction and a second direction that is a direction opposite to the first direction, in which the friction adjustment sheet is adhered to the tubular product such that friction in sliding the elastic garment from the first end portion side to the second end portion side is smaller than friction in sliding the elastic garment from the second end portion side to the first end portion side.

(15) A donning method for an elastic garment including: a donning process in which the elastic garment rolled up around the donning assistance tool by the donning preparation method according to (11) is donned on a hand or a foot.

### Effects of Invention

A user uses the donning assistance tool disclosed in the present application in combination with a commercially available plastic bottle and the like, and thus, the user can don the elastic garment. Therefore, it is unnecessary to carry a tubular product that supports the elastic garment, and thus, the convenience to the user who needs the elastic garment is improved.

### BRIEF DESCRIPTION OF DRAWINGS

FIG. 1 shows substitution photographs for drawings, which are photographs showing the example use of Magnide, a commercially available product.

FIG. 2 shows substitution photographs for drawings, which are photographs showing the example use of Stocking Donner, a commercially available product.

FIG. 3 shows substitution photographs for drawings, which are photographs showing the example use of Doff N′Donner, a commercially available product.

FIG. 4A is a schematic diagram showing the outline of a donning assistance tool 1.

FIG. 4B is a schematic diagram for explaining an exemplary modification of a ring-forming member 2 and a connecting body 3 of the donning assistance tool 1.

FIG. 4B-1 is an exemplary modification of the ring-forming member 2 and the connecting body 3 of the donning assistance tool 1, showing a schematic diagram for explaining a cut portion.

FIG. 4B-2 is a schematic diagram for explaining an exemplary modification of the ring-forming member 2 and the connecting body 3 of the donning assistance tool 1.

FIG. 4C shows schematic diagrams of the edge of the ring-forming member 2 in which the ring-forming member 2 is viewed from the extending direction of the through hole 22.

FIG. 5 shows substitution photographs for drawings, which is a photograph explaining the outline of a friction adjustment sheet 4.

FIG. 6 is a cross-sectional view showing a friction adjustment sheet 4 shown in FIG. 5 taken along Arrow x-x.

FIG. 7 shows substitution photographs for drawings, which are photographs for explaining the outline of a donning assistance kit according to a second embodiment.

FIG. 8 shows substitution photographs for drawings, showing the operation procedures of the processes of the donning preparation method.

FIG. 9 shows substitution photographs for drawings, showing the operation procedures of the processes of the donning preparation method.

FIG. 10 shows substitution photographs for drawings, showing the operation procedures of the processes of the donning preparation method.

FIG. 11 shows substitution photographs for drawings, showing the operation procedures of the processes of the donning preparation method.

FIG. 12 shows substitution photographs for drawings, showing the operation procedures of the processes of the donning preparation method.

FIG. 13 shows substitution photographs for drawings, showing the operation procedures of the processes of the donning preparation method.

FIG. 14 shows a substitution photograph for a drawing, showing a photograph of the donning processes of a donning method.

FIG. 15 shows substitution photographs for drawings, showing the donning assistance tool 1 prepared in Example 1.

FIG. 16 shows substitution photographs for drawings, showing a donning assistance tool 1 prepared in Example 2.

FIG. 17 shows substitution photographs for drawings, showing photographs of the rolling up operation of the end portion of the elastic garment and the rolling up operation of the elastic garment with respect to a donning assistance tool in the diameters, 30 mm and 10 mm, of the ring-forming member around which the end portion of the elastic garment rolled, and a donning assistance tool in the diameter, 20 mm, of the ring-forming member.

FIG. 18 shows substitution photographs for drawings, showing photographs of the difference in effects between the case of using and not using the donning assistance tool.

FIG. 19 shows substitution photographs for drawings, showing photographs of donning preparation situations in donning the elastic garment using a donning assistance tool, which is a commercially available product.

FIG. 20 shows substitution photographs for drawings, showing photographs of the difference in effects between the case of using the donning assistance tool, which is a commercially available product, and the case of using the donning assistance tool, which is prepared in Example 2.

### DESCRIPTION OF EMBODIMENTS

In the following, a donning assistance tool (in the following, sometimes simply referred to as a “donning assistance tool”) and a donning assistance kit for an elastic garment, and a donning preparation method (in the following, sometimes simply referred to as a “donning preparation method”) and a donning method for an elastic garment, which are disclosed in the present application, will be described in detail. Moreover, the positions, sizes, ranges, and the like of structures shown in the drawings may sometimes not show the actual positions, sizes, ranges, and the like in order to facilitate understanding. For this reason, the disclosure of the present application is not necessarily limited to the positions, sizes, ranges, and the like disclosed in the drawings.

Moreover, in the present specification, interpretations like (1) to (3) below are made.
(1) The numerical value ranges using the term “to” mean ranges including the numerical values written before and after the term “to” as the lower limit and the upper limit.
(2) The numerical value, the numerical value range, and qualitative representations (e.g., representations of the terms “equal” and “the same”) show numerical values, numerical value ranges, and properties including errors typically permitted in the relevant technical fields.
(3) In the case of writing the term “approximately xx”, shapes grasped as nearly xx are included, in addition to accurate shapes xx.

### Embodiment of Donning Assistance Tool

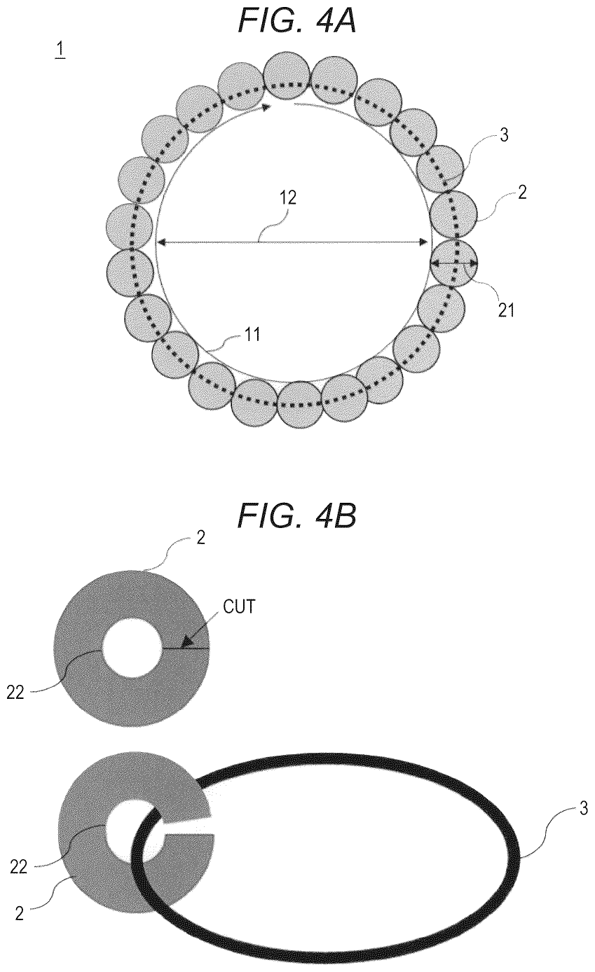

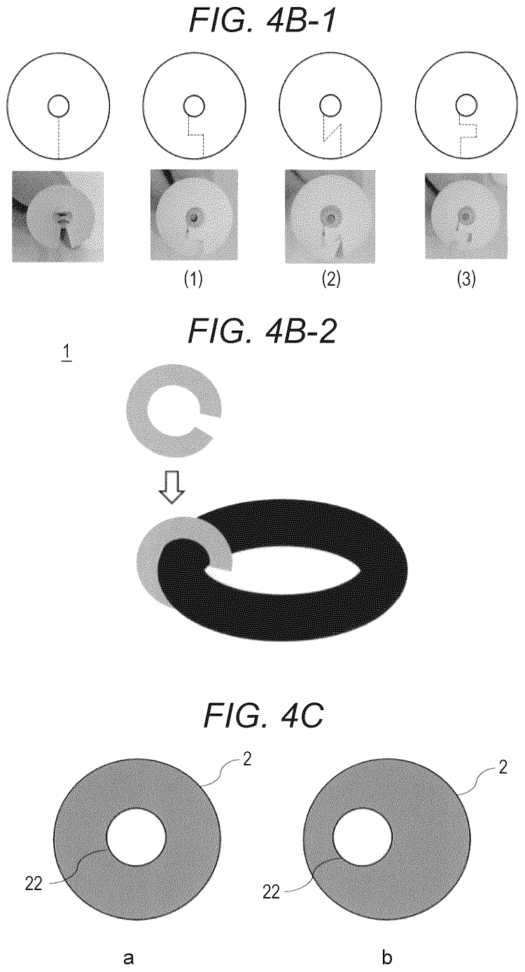

Referring to FIG. 4A, an embodiment of a donning assistance tool 1 will be described. FIG. 4A is a schematic diagram showing the outline of the donning assistance tool 1.

The donning assistance tool 1 according to the embodiment includes a plurality of ring-forming members 2 and a connecting body 3 that is made of a stretchable and contractible material and that connects ring-forming members 2. The ring-forming member 2 has a through hole into which the connecting body 3 is inserted. By inserting the connecting body 3 into the through hole, the ring body is formed. The formed ring body then functions as the donning assistance tool 1. In the following, reference sign 1 is also imparted to the ring body 1.

In performing a donning preparation method (details will be described later), a user places the donning assistance tool 1 on an elastic garment put on a tubular product, and moves the donning assistance tool 1 along the tubular product while rotating the donning assistance tool 1, and then the elastic garment is rolled around the donning assistance tool 1. As described above, the donning assistance tool 1 is rotated along the tubular product. For this reason, preferably, the ring-forming member 2 is a perfect spherical shape. However, the ring-forming member 2 is not limited to a spherical shape, as long as the shape is in the range in which the ring-forming member 2 rotates. For example, the ring-forming member 2 may be in a polyhedron with a number of faces in a degree allowing smooth rotation, an elliptic shape nearly a sphere, and a cylindrical shape. Moreover, dimples may be formed on the surface of the ring-forming member 2, like a golf ball. However, since the elastic garment has a risk of runs, in the case in which projections and depressions are formed on the surface of the ring-forming member 2, desirably, projections and depressions are formed to provide a smooth surface. The shape of the ring-forming member 2 may be all the same, or may be combined with the shapes exemplified above.

Preferably, a size 21 of the ring-forming member 2 ranges from 10 mm to 30 mm. The ring-forming member 2 is an operating part in rolling up the elastic garment. When the size 21 of the ring-forming member 2 is smaller than 10 mm, the operating part becomes small, degrading the operability of the rolling up operation of the elastic garment. Moreover, when the size 21 of the ring-forming member 2 is smaller than 10 mm, the number of rotations for rolling up the elastic garment in the same length is increased, leading to complications of the rolling up operation of the elastic garment. On the other hand, when the size 21 of the ring-forming member 2 is greater than 30 mm, the length removed from the tubular product becomes longer in the rolling up of the end portion of the elastic garment on the donning assistance tool 1, necessitating a larger force for removal. Therefore, in the case of a user who is specifically an elderly person, the operation becomes difficult.

Moreover, as described later, regarding the size of the ring body 1 that is the donning assistance tool 1, preferably, the diameter (inner diameter) ranges from 40 mm to 130 mm. When the size 21 of the ring-forming member 2 is greater than 30 mm, the number of the ring-forming members 2 constituting the ring body 1 becomes small. As a result, the number of the ring-forming members 2 contacting the elastic garment becomes small (the spacing for contact becomes large), leading to difficulty in evenly rolling up the elastic garment around the donning assistance tool 1.

Moreover, in the present specification, “the size of the ring-forming member” means the diameter in the case in which the ring-forming member 2 is a sphere. In the case in which the ring-forming member 2 has a shape other than a sphere, the longest length in the lengths of the ring-forming member 2 measured with vernier calipers and the like only has to be set to “the size of the ring-forming member”.

The number of the ring-forming members 2 only has to be designed suitably for the diameter of the ring body 1 to be designed and the size 21 of the ring-forming member 2. Moreover, the size 21 of the plurality of ring-forming members 2 constituting the ring body 1 only has to be all the same. Alternatively, in relation to adjusting the size of the ring body 1, the size 21 of a part of the ring-forming member 2 may be changed. The number of the ring-forming members 2 that change their size 21 is unlimited, specifically, as long as the number is within the range that does not degrade the workability and functionality of the donning assistance tool 1. For example, one, two, three, four, and so on. Moreover, in the case of changing the size 21 of two or more ring-forming members 2, preferably, the ring-forming members 2 of which the size 21 is changed are placed at nearly regular intervals so as not to vary the properties of the donning assistance tool 1.

The material of the ring-forming member 2 is not specifically limited, as long as the elastic garment is allowed to roll up around the donning assistance tool 1 by moving and rotating the donning assistance tool 1 along the tubular product. Examples of the material include resin, rubber, metal, wood, and the like. Moreover, since the ring-forming member 2 contacts the elastic garment, it is assumed that it is difficult to roll up the elastic garment when the surface is slippery. For this reason, desirably, the elastic garment is not slippery on the surface of the ring-forming member 2. Examples of preferable materials include resin, including silicon resin and vinyl chloride resin, and rubbers, including silicone rubber and nylon rubber. Moreover, it is unnecessary to form the ring-forming member 2 with a single material. For example, the ring-forming member 2 may be formed by coating a publicly known slip-resistance material, including a silicon spray, on the surface of a relatively slippery material. Moreover, the ring-forming member 2 may be solid or hollow. The determination of solid or hollow only has to be made to provide a desired weight of the donning assistance tool 1, suitable for the size of the donning assistance tool 1 and the material of the ring-forming member 2 to be used.

The connecting body 3 has no limitations specifically, as long as materials that can stretch the ring body 1, formed by being inserted into a through hole formed on the ring-forming member 2. The connecting body 3 only has to be formed of a material with stretching and contraction properties, including rubber, which does not impose limitations.

The connecting body 3 only has to be inserted into the through hole formed on the ring-forming member 2 such that the ring-forming member 2 is allowed to or not allowed to rotate with respect to the connecting body 3. For example, all the ring-forming members 2 are inserted into the connecting body 3 such that the ring-forming members 2 are rotatable with respect to the connecting body 3. Alternatively, the ring-forming members 2 may be fixed to the connecting body 3 such that a part of the ring-forming members 2 is rotatable with respect to the connecting body 3, and the other ring-forming members 2 are not allowed to rotate with respect to the connecting body 3. Moreover, in the case in which the ring-forming members 2 are rotatable with respect to the connecting body 3, the ring-forming members 2 rotate with respect to the connecting body 3 and are movable along the connecting body 3 in stretching the donning assistance tool 1. For this reason, in the case in which two or more of the ring-forming members 2 are fixed so as not to rotate with respect to the connecting body 3, the ring-forming members 2 to be fixed may be fixed to the connecting body 3, being placed at nearly regular intervals. In the case in which the ring-forming member 2 is fixed to the connecting body 3 at nearly regular intervals, the adjacent fixed ring-forming members 2 function as stoppers in moving the rotatable ring-forming member 2 placed between the fixed ring-forming members 2 along the connecting body 3. Therefore, in stretching the donning assistance tool 1, it is possible to stop the ring-forming members 2 rotatably inserted into the connecting body 3 from being placed unevenly at one place. Furthermore, alternatively, all the ring-forming members 2 may be fixed to the connecting body 3 so as not to be rotatable with respect to the connecting body 3.

Preferably, regarding the size of the ring body 1 in a non-stretched state, a diameter (the inner diameter of the ring body) 12 ranges from 40 mm to 130 mm, in placing the ring body 1 such that a virtual line 11 inscribed in the ring-forming members 2 constituting the ring body 1 makes a circle. In the case of providing the donning assistance tool 1, it is possible to provide various donning assistance tools 1 varied in size, having diameters 12, 40 mm, 50 mm, 60 mm, 70 mm, 80 mm, 90 mm, 100 mm, 120 mm, and 130 mm, for example. The donning assistance tool 1 in an appropriate size only has to be used, suitable for the part (hand or foot) of the body of a user who dons, the form of a user, and the size of the tubular product.

Moreover, in the present specification, the term “the non-stretched state of the ring body” means a state in which the ring-forming members 2 constituting the ring body 1 contact each other and there is no spacing between the ring-forming members 2 adjacent to each other. As described above, the size 21 of a part of the ring-forming members 2 may be changed. In this case, the diameter 12 of a virtual circle to which the most ring-forming members 2 can contact only has to be set to the diameter 12 of the ring body 1.

As described in the donning preparation method, described later, a user places the donning assistance tool 1 on the elastic garment put on the tubular product, moves the donning assistance tool 1 while rotating the donning assistance tool 1 along the tubular product, and rolls up the elastic garment on the donning assistance tool 1. At this time, the donning assistance tool 1 disclosed in the present application is stretchable and contractible, i.e., the donning assistance tool 1 is movable while changing the shape of the donning assistance tool 1 along the shape of the tubular product in moving the donning assistance tool 1 along the tubular product. Therefore, in the donning preparation method disclosed in the present application, it is possible to use a commercially available plastic bottle and the like, not a dedicated tubular product.

As described above, since the donning assistance tool 1 can change its shape, the plastic bottle may have a nearly cylindrical shape or a nearly columnar shape. However, from the viewpoint of ease of donning the elastic garment, preferably, the plastic bottle has a nearly cylindrical shape. Moreover, some of the commercially available plastic bottles are made of an extremely soft material. In regard to this, in performing the donning preparation method, a plastic bottle that is relatively hard and has a round shape is preferable. An example of a relatively hard plastic bottle includes a carbonated beverage plastic bottle. The sizes of most commercially available carbonated beverage plastic bottles are 500 mL and 1.5 L. The diameter of the 500 mL plastic bottle varies, depending on the type, and is approximately 69 mm. The diameter of the 1.5 L plastic bottle varies, as well, depending on the type, and is approximately 90 mm. When the diameter of the donning assistance tool 1 is too large compared with the diameter of the plastic bottle, the operability degrades. In the case in which the diameter of the donning assistance tool 1 is smaller, the donning assistance tool 1 stretches and can move along the plastic bottle. Therefore, the diameter of the donning assistance tool (ring body) I used for the plastic bottle in 500 mL preferably ranges from approximately 40 mm to 70 mm. The diameter of the donning assistance tool 1 used for the plastic bottle in 1.5 L preferably ranges from approximately 40 mm to 90 mm. Moreover, since the sizes of plastic bottles easily obtainable are 500 mL and 1.5 L, the diameter of the donning assistance tool (ring body) 1 preferably ranges from approximately 40 mm to 90 mm. However, there are some plastic bottles in the size (e.g., 2 L) greater than the size of 1.5 L. Therefore, as described above, the diameter of the donning assistance tool (ring body) I may range from approximately 40 mm to 130 mm.

Examples of the elastic garment include a knee sock below the knee, a stocking pulled up to the thigh above the knee, and a sleeve used for the arm. Among them, in the case in which the donning assistance tool 1 is used for donning a stocking, it is necessary to move the donning assistance tool 1 from the toe to the thigh. In this case, the connecting body 3 is preferably stretchable and contractible at least approximately three to four times based on the length of the connecting body 3 in the non-stretched ring body 1. As a material for forming the connecting body 3, rubber and the like with specifically high stretching performance only have to be used.

Some specific embodiments of the donning assistance tool 1 are described with reference to FIG. 4A. However, various modifications are possible without being limited to the embodiment, as long as the function of the donning assistance tool 1 disclosed in the present application is obtained. Next, various exemplary modifications adoptable to the donning assistance tool 1 will be described.

### (Ring-Forming Member 2 and Connecting Body 3)

Referring to FIG. 4B, an exemplary modification of the ring-forming member 2 and the connecting body 3 will be described. FIG. 4B is a schematic diagram for explaining the outline of the exemplary modification. In the example shown in FIG. 4A, a through hole is formed in the ring-forming member 2, the connecting body 3 is inserted into the through hole, and the donning assistance tool 1 is formed. In this case, in order to form the donning assistance tool 1, it is necessary to form a knot at the end portions of the connecting body 3, or it is necessary to fix the end portions of the connecting body 3 to the through hole of a given ring-forming member 2 with an adhesive and the like.

On the other hand, as shown in FIG. 4B, in the donning assistance tool 1, the connecting body 3 in a seamless ring shape and the ring-forming member 2 formed with a cut may be used. In the example shown in FIG. 4B, the cut portion is opened to deform the ring-forming member 2 such that an approximately C-shape is formed when viewed from the extending direction of the through hole 22, and thus, the connecting body 3 can be inserted into the through hole 22 of the ring-forming member 2 through the cut. The cut portion may be adhered with an adhesive and the like so as not to separate after insertion into the connecting body 3, or may have a structure that is fittable and retainable (a specific example will be described later). In the case in which the fittable and retainable structure maintains the retention state with force in using the donning assistance tool 1, whereas the structure releases the retention state when a force greater than the force when the donning assistance tool 1 is used, the ring-forming member 2 is detachable with respect to the connecting body 3. In actual use of the donning assistance tool 1, the connecting body 3 stretches. In the example shown in FIG. 4A, a large force is applied to the knot or the adhered portion of the connecting body 3, and the connecting body 3 is prone to be cut at the knot or the adhered portion. On the other hand, in the example shown in FIG. 4B, since the connecting body 3 is seamless, the risks of cuts caused by the knot or the adhered portion of the connecting body 3 become small. Furthermore, in the case in which the ring-forming member 2 can be attached so as to be detachable with respect to the connecting body 3, the ring-forming member 2 is added or removed depending on the use situations, and thus, the inner diameter of the ring body 1 can be adjusted.

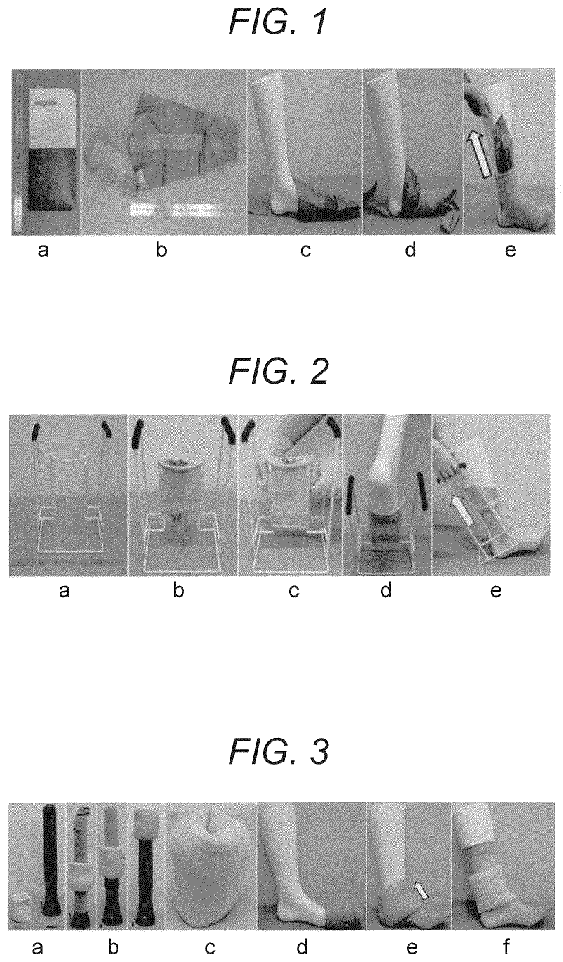

Next, referring to FIG. 4B-1, specific examples of the cut portion will be described in detail. In the example shown in FIG. 4B, the cut formed on the ring-forming member 2 is approximately linearly formed to reach from the through hole 22 to the outer edge of the ring-forming member 2 when the through hole 22 is viewed from the front. In other words, the cut is formed approximately in a flat shape on the ring-forming member 2. Alternatively, as shown in (1) to (3) of FIG. 4B-1, the cut formed on the ring-forming member 2 may be formed non-linearly when the through hole 22 is viewed from the front (for comparison, the embodiment of the linear cut depicted in FIG. 4B is shown on the left side of FIG. 4B-1). In other words, the cut may be formed on the ring-forming member 2 so as to make a pair of fittable and retainable structures. Moreover, a pair of fittable and retainable structures may include a curved surface portion. However, from the viewpoint of hardly removing the fittable and retainable structure when an external force is applied, more preferably, the pair of fittable and retainable structures is formed in combination with a flat portion. In the example shown in (1) to (3) of FIG. 4B-1, the cut of the ring-forming member 2 is formed in a non-flat shape, and thus the fittable and retainable structure is formed. Thus, it is difficult to form a continuous space from the through hole 22 toward the outer edge of the ring-forming member 2, when an external force is applied to the ring-forming member 2. Therefore, it is possible to obtain the effect that the ring-forming member 2 does not easily drop off from the connecting body 3.

Moreover, in the example shown in FIG. 4B, the size of the through hole 22 of the ring-forming member 2 is greater than the connecting body 3. Therefore, in the example shown in FIG. 4B, when the ring body 1 is not used (when the connecting body 3 is not stretched), the ring-forming members 2 attached to the connecting body 3 are rotatable and movable with respect to the connecting body 3. Alternatively, as shown in FIG. 4B-2, the ring-forming members 2 may be formed such that adjustment is performed such that the size of the through hole 22 of the ring-forming member 2 and the size of the outer diameter of the connecting body 3 are almost equal, and thus, the ring-forming members 2 are not easily rotated with respect to the connecting body 3, or the ring-forming members 2 are not allowed to rotate and move, when the ring body 1 is not used (when the connecting body 3 is not stretched). Moreover, since the connecting body 3 is stretched when the ring body 1 is used, the size of the outer diameter of the connecting body 3 when stretched is smaller than the size when not used. Therefore, also in the example shown in FIG. 4B-2, when the ring body 1 is used (when the connecting body 3 is stretched), the ring-forming members 2 are rotatable and movable with respect to the connecting body 3. Moreover, although not shown in the drawing, as described above, the size of the outer diameter of the connecting body 3 becomes small when stretched. Therefore, as long as the ring-forming members 2 are rotatable and movable with respect to the connecting body 3 when the ring body 1 is used (when the connecting body 3 is stretched), the size of the connecting body 3 when the ring body 1 is not used (when the connecting body 3 is not stretched) may be further greater than the example shown in FIG. 4B-2 (in the following, referred to as “the exemplary modification of FIG. 4B-2”). It can be said that FIG. 4B-2 and the exemplary modification of FIG. 4B-2, i.e., the ring-forming member 2 and the connecting body 3 are formed such that the ring-forming members 2 are rotatable and movable with respect to the connecting body 3 when the connecting body 3 is stretched, whereas the rotation and moving properties of the ring-forming member 2 with respect to the connecting body 3 are degraded when the connecting body 3 is not stretched, as compared with the time when stretched.

In the examples shown in FIG. 4B to FIG. 4B-2, the effects described below are obtained and exerted in combination.
(1) Even in the case in which any one of the ring-forming member 2 and the connecting body 3 is degraded, easy replacement is possible.
(2) The ring-forming member 2 and the connecting body 3 are easily detached. Therefore, in the case in which the ring body 1 is cleaned as it is, it is possible to neatly clean the parts that are not easily cleaned.
(3) The case is assumed that the connecting body 3 is prepared with rubber. The rubber is prone to degradation when the stretched state continues. However, in the examples shown in FIG. 4B to FIG. 4B-2, it is possible to attach the ring-forming member 2 in the non-stretched state of the connecting body 3. Therefore, it is possible to slow the degradation speed of the connecting body 3.
(4) A preparation method is assumed that the connecting body 3 is inserted into the through hole 22 of the ring-forming member 2 and the end portions of the connecting body 3 are finally tied. In this case, when the outer diameter of the connecting body 3 and the size of the through hole 22 are the same, the operation in which the connecting body 3 is inserted into the through hole 22 becomes difficult due to friction. Therefore, the outer diameter of the connecting body 3 inevitably becomes smaller than the size of the through hole 22. On the other hand, in the method shown in FIG. 4B to FIG. 4B-2, the size of the outer diameter of the connecting body 3 can be made greater than the preparation method, in which the connecting body 3 is inserted into the through hole 22. The greater the size of the outer diameter of the connecting body 3 becomes, the greater the strength when stretched. Therefore, in the examples shown in FIG. 4B to FIG. 4B-2, the connecting body 3 is not easily cut when the ring body 1 is used.

### (Position of the Through Hole 22 in the Ring-Forming Member 2)

FIG. 4C is a schematic diagram of the ring-forming member 2 when viewed from the extending direction of the through hole 22. As shown in a of FIG. 4C, the through hole 22 only has to be formed so as to pass approximately the center of the ring-forming member 2. In the case in which the through hole 22 is formed so as to pass approximately the center of the ring-forming member 2, the donning assistance tool 1 can be moved while smoothly rotating along the tubular product or a donning target, in performing the donning preparation method and the donning method. On the other hand, as shown in b of FIG. 4C, the through hole 22 may be formed at a position off-centered from approximately the center of the ring-forming member 2. In the ring-forming member 2 having the off-centered through hole shown b of FIG. 4C, the length of the through hole is shorter than that of the non-off-centered ring-forming member 2. For this reason, it is possible to form the connecting body 3 with a much larger number of the ring-forming members 2 than the ring body 1 in the same diameter.

Various embodiments and various adoptable exemplary modifications of the donning assistance tool 1, exemplified as described above, may be combined with each other. For example, the ring-forming members 2, which are different in sizes, shapes, and positions of the through hole 22 may be combined. Moreover, the ring-forming member 2 with the cut shown in FIG. 4B may be combined with the ring-forming member 2 with no cut.

### First Embodiment of the Donning Assistance Kit

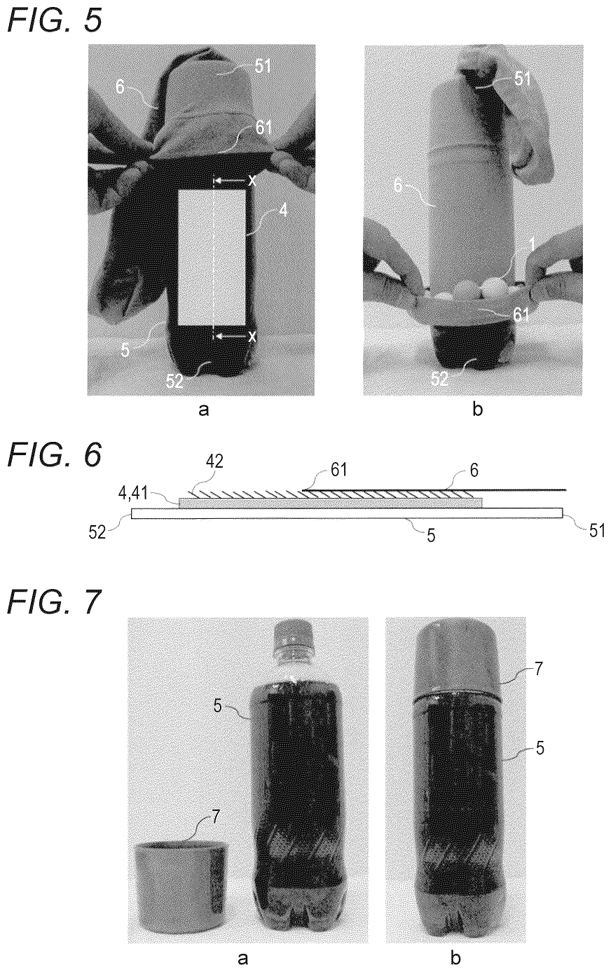

Next, referring to FIG. 5 and FIG. 6, a donning assistance kit according to a first embodiment will be described. The donning assistance kit according to the first embodiment includes any one of the donning assistance tools 1 described in the embodiment of the donning assistance tool and a friction adjustment sheet 4. FIG. 5 is a substitution photograph for drawings showing the outline of the friction adjustment sheet 4. FIG. 6 is a cross-sectional view showing the friction adjustment sheet 4 shown in FIG. 5 taken along Arrow x-x.

As shown in a of FIG. 5, the friction adjustment sheet 4 used being adhered to the tubular product 5. The friction adjustment sheet 4 is designed such that the friction when an elastic garment 6 is moved in the state in which the elastic garment 6 is contacted is varied between a first direction and a second direction that is the direction opposite to the first direction. More specifically, in the donning preparation method, first, the end portion 61 of the elastic garment 6 is attached while sliding from a first end portion side 51 of the tubular product 5 to a second end portion side 52 (see a of FIG. 5). At this time, preferably, the frictional resistance between the elastic garment 6 and the tubular product 5 is smaller.

Subsequently, the elastic garment 6 attached on the tubular product 5 is rolled up around the donning assistance tool 1 while placing the end portion 61 of the elastic garment 6 attached on the tubular product 5 on the donning assistance tool 1 and moving the donning assistance tool 1 from the second end portion side 52 to the first end portion side 51 of the tubular product 5 (see b of FIG. 5). When the elastic garment 6 is slippery from the second end portion side 52 to the first end portion side 51, there might be risks in which the elastic garment 6 slides on the tubular product 5, causing wrinkles in the elastic garment 6 in moving the donning assistance tool 1 from the second end portion side 52 to the first end portion side 51. Therefore, in order not to easily slide the elastic garment 6 from the second end portion side 52 to the first end portion side 51, preferably, the frictional resistance between the elastic garment 6 and the tubular product 5 in moving in that direction becomes larger.

The friction adjustment sheet 4 is designed such that the first direction is different from in the second direction that is the direction opposite to the first direction. For this reason, the friction adjustment sheet 4 only has to be adhered to the tubular product 5 such that the friction in moving the elastic garment from the first end portion side 51 to the direction of the second end portion side 52 becomes small. Schemes for varying the first direction from the second direction are not limited specifically. In the example shown in FIG. 6, fibers 42 in inclination are placed on the base material 41 of the friction adjustment sheet 4, and thus, the friction in moving the elastic garment 6 from the first end portion side 51 to the second end portion side 52 is different from the friction in moving the elastic garment 6 from the second end portion side 52 to the first end portion side 51.

The friction adjustment sheet 4 may be adhered to entirely on the surface of the tubular product 5, or may be adhered to a part of the surface. In the case in which the friction adjustment sheet 4 is provided as a component of the donning assistance kit, the friction adjustment sheet 4 may be one, two, or more. In the case in which two or more of the friction adjustment sheets 4 are provided, the sizes of the individual friction adjustment sheets 4 may be the same or may be different. Moreover, the section adhering to the tubular product 5 may be made of a detachable adhesive substance, allowing the friction adjustment sheet 4 to be reused.

### Second Embodiment of the Donning Assistance Kit

Next, referring to FIG. 7, the donning assistance kit according to a second embodiment will be described. The donning assistance kit according to the second embodiment includes any one of the donning assistance tools 1 described in the embodiment of the donning assistance tool and a cup 7.

As shown in a of FIG. 7, a commercially available plastic bottle has a small spout side. Of course, it is possible to perform the donning preparation method, described later, without using the cup 7. However, since the cup 7 is inserted into the spout of the plastic bottle to substantially prolong the length of the tubular product 5, an effect of prolonging the elastic garment 6 to be donned is obtained and exerted. Moreover, although the details will be described in the donning preparation method, there is a case in which in rolling up the elastic garment 6 on the donning assistance tool 1, the first end portion side 51 and the second end portion side 52 of the tubular product 5 are reversed upside down, and the first end portion side 51 (the spout side in the example shown in FIG. 7) is contacted with a rack and the like. In the case in which the cup 7 is inserted, it is possible to obtain the effect that stability is improved more than the case in which the spout of the plastic bottle is directly contacted with the rack and the like.

In the examples shown in a and b of FIG. 7, an example of the size the same as the size of the plastic bottle is shown, in inserting the cup 7 into the spout of the plastic bottle that is the tubular product 5. As described above, since the diameter of the plastic bottle in 500 mL is approximately 69 mm and the diameter of the plastic bottle in 1.5 L is approximately 90 mm, desirably, the outer diameter of the cup 7 is approximately 69 mm or approximately 90 mm. Moreover, the friction adjustment sheet 4 and the cup 7 described in the donning assistance kit according to the first and the second embodiments may be used in combination.

### Third Embodiment of the Donning Assistance Kit

Next, although not shown in the drawing, the donning assistance kit according to a third embodiment will be described. The donning assistance kit according to the third embodiment includes any one of the donning assistance tools 1 described in the embodiment of the donning assistance tool and a tubular product 5 in the outer diameter ranging from 36 mm to 143 mm. The outer diameter of the tubular product 5 is the same as the diameter of the ring body 1 in the non-stretched state, or is in the range ±10% of the diameter of the ring body 1 in the non-stretched state.

The donning assistance tool 1 disclosed in the present application allows donning the elastic garment using a commercially available plastic bottle. For this reason, a product to be carried to a place, for example, away from home, is only the donning assistance tool 1 (the friction adjustment sheet 4 and/or the cup 7, as necessary), and it is unnecessary to carry the tubular product 5, which is bulky. On the other hand, the donning assistance tool 1 disclosed in the present application can also be used at home or in a hospital room, and the like. In this case, the operability of a user is improved when the tubular product 5 is suitable for the size of the donning assistance tool 1. As described above, the donning assistance tool 1 is moved while rotating along the tubular product 5, and the elastic garment is rolled up around the donning assistance tool 1. For this reason, although the inner diameter of the donning assistance tool 1 preferably matches the outer diameter of the tubular product 5, the donning assistance tool 1 can be preferably used as long as within a range of ±10% errors.

### Embodiment of the Donning Preparation Method

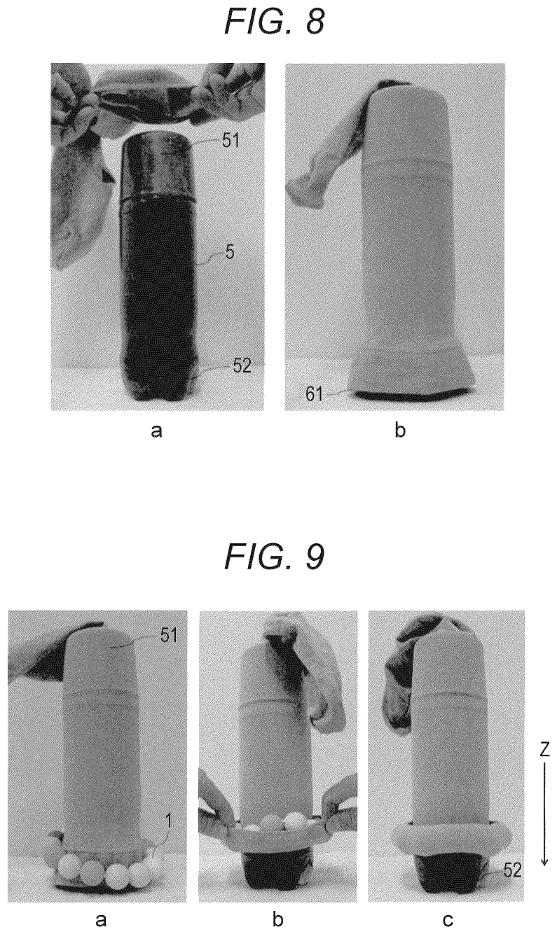

Referring to FIG. 8 to FIG. 13, the outline of an embodiment of the donning preparation method will be described. FIG. 8 to FIG. 13 are substitution photographs for drawings showing the operation procedures of the processes of the donning preparation method. The donning preparation method is performed using any one of the donning assistance tools 1 described in the embodiment of the donning assistance tool and the tubular product 5. In the example shown in FIG. 8 to FIG. 13, the plastic bottle (Pepsi-Cola) in 500 mL is used as the tubular product 5, and the cup 7 is inserted. However, a dedicated tubular product 5 may not be used. Moreover, the cup 7 may not be used. Moreover, in FIG. 8 to FIG. 13, reference signs are sometimes partially omitted to avoid complications in the drawings.

The donning preparation method includes an elastic garment end portion attaching process, an elastic garment rolling up process, an elastic garment re-attaching process, and an attached elastic garment re-rolling up process.

FIG. 8 shows photographs of the outline of the elastic garment end portion attaching process. In the elastic garment end portion attaching process, the end portion 61 of the elastic garment 6 is attached to the tubular product 5, while sliding from the first end portion side 51 to the second end portion side 52 of the tubular product 5.

FIG. 9 shows photographs of the outline of the elastic garment rolling up process. In the elastic garment rolling up process, the donning assistance tool 1 is placed on the end portion 61 of the elastic garment 6 attached on the tubular product 5, the donning assistance tool 1 is moved while rotating from the second end portion side 52 to the first end portion side 51 of the tubular product 5 along the tubular product 5, and the elastic garment 6 is rolled up around the donning assistance tool 1. Moreover, in FIG. 9, the direction of rolling up the elastic garment 6 using the donning assistance tool 1 is the direction opposite to a gravity direction Z. Alternatively, although not shown in the drawing, the elastic garment rolling up process may be performed such that the tubular product 5 is laid vertically (a line connecting the first end portion side 51 to the second end portion side 52 is the direction nearly orthogonal to the gravity direction). Furthermore, alternatively, after the end portion 61 of the elastic garment 6 is rolled up around the donning assistance tool 1, as shown in FIG. 10, the first end portion side 51 and the second end portion side 52 may be reversed and inverted to roll up the elastic garment 6 while moving the donning assistance tool 1 from the second end portion side 52 to the first end portion side 51 in the direction (the gravity direction Z). In the examples shown in FIG. 10, the direction of moving the donning assistance tool 1 with a predetermined weight is approximately the gravity direction. Therefore, in the examples shown in FIG. 10, it is possible to obtain the effect that a user can perform the elastic garment rolling up process with a small force.

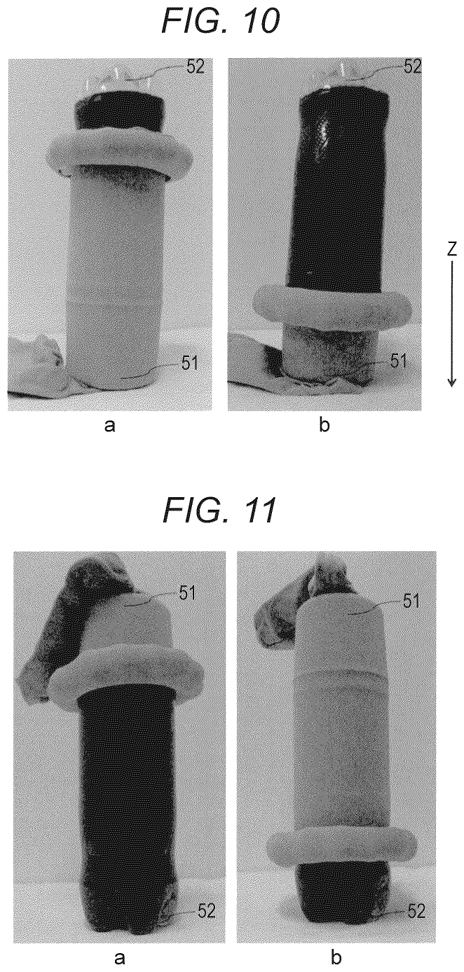

FIG. 11 shows photographs of the outline of the elastic garment re-attaching process. In the elastic garment re-attaching process, the donning assistance tool 1, around which the elastic garment 6 is rolled up, is slid from the first end portion side 51 to the second end portion side 52, and the elastic garment 6 is again attached to the tubular product 5. Moreover, in the elastic garment rolling up process, as shown in FIG. 10, in the case in which the first end portion side 51 and the second end portion side 52 are reversed and inverted, the tubular product 5 only has to be again reversed and inverted such that the first end portion side 51 is located on the upper side of the gravity direction Z before the elastic garment re-attaching process is performed.

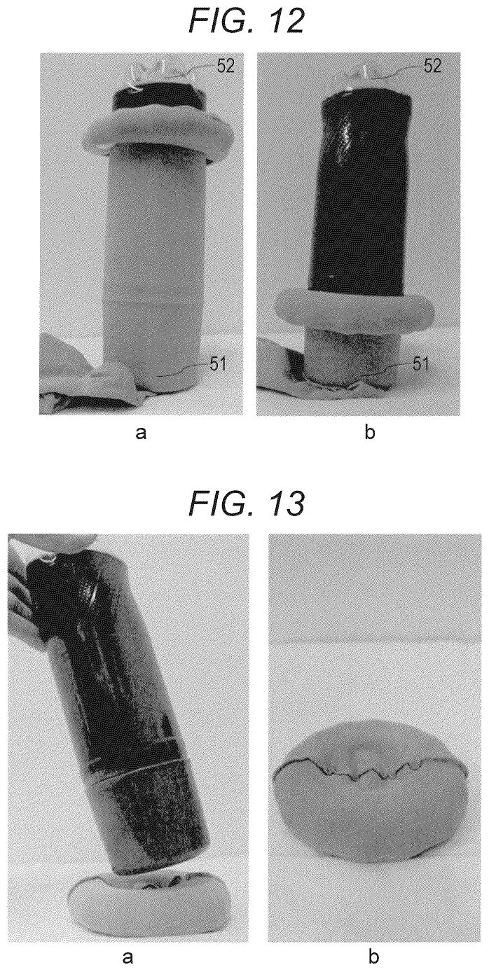

FIG. 12 shows photographs of the outline of the attached elastic garment re-rolling up process. In the attached elastic garment re-rolling up process, the donning assistance tool 1 is moved while rotating from the second end portion side 52 to the first end portion side 51 of the tubular product 5 along the tubular product, and the elastic garment 6 again attached to the donning assistance tool 1 is rolled up. regarding the orientation of the tubular product 5 in the attached elastic garment re-rolling up process, similarly to the elastic garment rolling up process, the direction in which the elastic garment 6 is rolled up with the donning assistance tool 1 may be any direction of the direction the same as the gravity direction Z, the reverse direction, and the direction nearly orthogonal to the gravity direction Z. However, the rolling up direction is set to the direction the same as the gravity direction Z, and thus, a user can perform the attached elastic garment re-rolling up process with a small force.

FIG. 13 shows photographs after the elastic garment 6 is rolled up around the donning assistance tool 1. After the elastic garment 6 is entirely rolled up around the donning assistance tool 1, the donning assistance tool 1 around which the elastic garment 6 is rolled up only has to be removed from the tubular product 5.

### (Optional Additional Matters Adoptable to the Embodiment of the Donning Preparation Method)

### <Repeating the Processes>

In the donning preparation method, the elastic garment re-attaching process and the attached elastic garment re-rolling up process may be repeatedly performed after the attached elastic garment re-rolling up process. In the case in which the elastic garment 6 is longer than the length of the tubular product 5 for use, it is possible to roll up the elastic garment 6 entirely around the donning assistance tool 1.

### <Use of the Friction Adjustment Sheet>

In performing the donning preparation method, the friction adjustment sheet 4 may be adhered to the tubular product 5 such that the friction in sliding the elastic garment 6 from the first end portion side 51 to the second end portion side 52 becomes smaller than the friction in sliding the elastic garment 6 from the second end portion side 52 to the first end portion side 51. In the case in which the friction adjustment sheet 4 is used, the elastic garment 6 is slippery when being attached to the tubular product 5, whereas the elastic garment 6 is not easily slippery with respect to the tubular product 5 in rolling up the elastic garment 6 on the donning assistance tool 1.

Therefore, it is possible to obtain the effect that the elastic garment 6 is easily and evenly rolled up around the donning assistance tool 1.

The processes, <Repeating the processes> and <Use of the friction adjustment sheet>, may be performed in combination.

### Embodiment of the Donning Method for the Elastic Garment

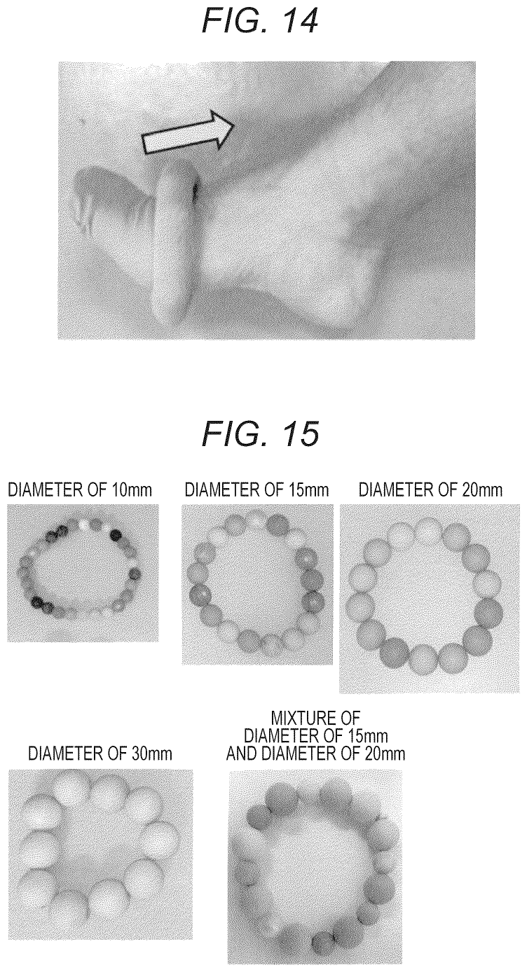

Referring to FIG. 14, the outline of an embodiment of the donning method for the elastic garment will be described. FIG. 14 is a substitution photograph for a drawing showing the donning process of the donning method. The donning method includes the donning process in which the elastic garment 6 is rolled up around the donning assistance tool 1 by the donning preparation method, which is donned on a hand or foot. As shown in FIG. 14, in the case in which the elastic garment 6 is donned on the foot, the donning assistance tool 1, around which the elastic garment 6 is rolled up, only has to be donned on the toe to don the elastic garment 6 while rotating the donning assistance tool 1 along the foot. Moreover, although not shown in the drawing, in the case in which the elastic garment 6 is donned on the hand, the elastic garment 6 may be donned such that the donning assistance tool 1 around which the elastic garment 6 is rolled up is donned on the fingertip, while rotating the donning assistance tool 1 along the arm.

The donning preparation method and the donning method are performed using the donning assistance tool 1 disclosed in the present application, and thus, it is possible to obtain the following effect.
(1) The size of the donning assistance tool 1 itself is smaller than that of the commercially available product shown in FIG. 1 to FIG. 3. The donning assistance tool 1 can be used for donning the elastic garment in combination with a commercially available plastic bottle and the like. For this reason, the convenience of carrying the donning assistance tool 1 by the user is improved.
(2) The donning assistance tool 1 is formed by connecting the ring-forming members 2 using the stretchable and contractible connecting body 3, and thus, the donning assistance tool 1 is deformable. Therefore, the following effects and the like are obtained in combination.
   In rolling up the elastic garment 6 on the donning assistance tool 1, the shape of the donning assistance tool 1 is changeable so as to follow the shape of the tubular product 5. For this reason, it is possible to use the tubular product 5 in a wide variety of shapes.
   In donning the rolled up elastic garment 6 on the user, the shape of the donning assistance tool 1 is changeable along the body shape of the user. For this reason, it is possible to reduce the occurrence of displacement in donning the elastic garment 6 on the user.
   When a material with a wide stretchable and contractible area is used for the connecting body 3, it is possible to don the elastic garment 6 on different sites from the arm to the thigh with a single donning assistance tool 1.
(3) The ring-forming member 2 is in a rotatable shape. For this reason, in donning the rolled up elastic garment on the user, it is also possible to don the elastic garment 6 while pressing the donning assistance tool 1 against the user. In this case, it is possible to don the elastic garment 6 on the user while imparting a massage effect to the user. Moreover, it is also possible to use the donning assistance tool 1 as a massage tool by inserting the donning assistance tool 1 itself into the foot or the arm of the user for pressing after donning the elastic garment 6 on the user. Therefore, the donning assistance tool 1 disclosed in the present application has different functions: the function of auxiliary assisting the donning of the elastic garment 6 and the massage function for the user.
(4) The donning assistance tool 1 is inserted into near the end portion 61 of the elastic garment 6 donned on the user, the donning assistance tool 1 is rotated and moved while rolling up the end portion 61 around the donning assistance tool 1, and thus, it is also possible to use the donning assistance tool 1 to doff the elastic garment 6. For example, in the case in which the doffed elastic garment 6 is again used, including the case in which the site where the elastic garment 6 is donned is examined in a hospital and the like, and the case in which the elastic garment 6 with a small smear is doffed and again donned as it is, it is possible to perform the donning method without starting over the donning preparation method. For this reason, the user's convenience is improved.
(5) In donning the elastic garment 6 rolled up around the donning assistance tool 1 on the user, the donning assistance tool 1 is moved, while rotating along the foot or the arm of the user, and thus, it is possible to don the elastic garment 6 on the user. As shown in an example and a comparative example, described later, with the use of the donning assistance tool 1 disclosed in the present application, it is possible to don the elastic garment 6 with a small traction force.
(6) As shown in an example and a comparative example, described later, in donning the elastic garment using the donning assistance tool 1 disclosed in the present application, it is possible to achieve the donning that the elastic garment is equally stretched.

In the following, the embodiment that is disclosed in the present application will be described in more detail with an example. In regard to this, this example is simply provided for explaining the embodiment. This is not limited or restricted to the scope of the inventions disclosed in the present application.

### EXAMPLE

### [Preparation of the Donning Assistance Tool 1]

The donning assistance tool 1 was prepared using the following material.

### Example 1 of the Donning Assistance Tool 1

### (1) Ring-Forming Member

Diameter: 10 mm, 15 mm, 20 mm, 30 mm
Material: silicon resin

Moreover, since silicon was not available for a material in 30 mm, a wooden product was used.

### (2) Connecting Body

For the connecting body, Operon rubber (trade name) was used.

The connecting body was inserted into the through hole of the ring-forming member 2, the end portions of the connecting body were tied, and the donning assistance tool 1 was prepared. FIG. 15 shows photographs of the prepared donning assistance tools 1. In the examples shown in FIG. 15, even in the case in which the donning assistance tool 1 was prepared using the ring-forming member 2 with the same diameter alone, the ring body was able to be formed with no gap. Meanwhile, the gap is possibly produced between the ring-forming members 2, depending on the diameter of the ring body. As shown in “the mixture of the diameters of 15 mm and 20 mm” in FIG. 15, in the case in which the donning assistance tools 1 were prepared by mixing the ring-forming members 2 with different diameters, it is possible to obtain the effect of eliminating the gap between the ring-forming members 2.

### Example 2 of the Donning Assistance Tool 1

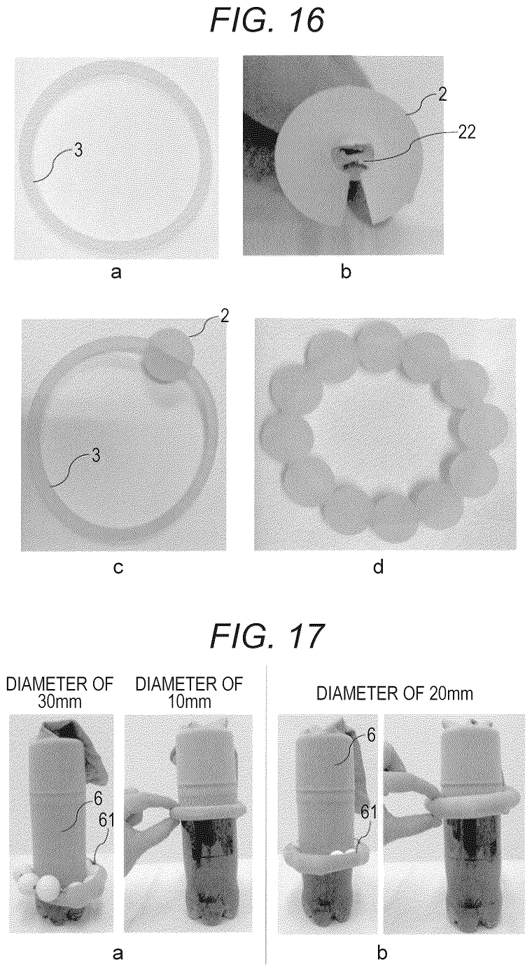

Referring to FIG. 16, Example 2 of the donning assistance tool 1 will be described. In Example 2, seamless silicone rubber shown in a of FIG. 16 was used for the connecting body 3. The ring-forming member 2 is made of silicon, and has a cut as shown in b of FIG. 16. The connecting body 3 was inserted into the through hole 22 of the ring-forming member 2 while opening the cut (c of FIG. 16). The plurality of the ring-forming members 2 was inserted into the connecting body 3 while opening the plurality of the ring-forming members 2, and the donning assistance tool 1 was prepared as shown in d of FIG. 16.

### [Performing the Donning Preparation Method and the Donning Method]

FIG. 8 to FIG. 14 are photographs of performing the donning preparation method and the donning method using the ring-forming member with a diameter of 20 mm (the diameter of the ring body was 70 mm) in the prepared donning assistance tools 1. As shown in FIG. 8 to FIG. 14, it was confirmed that the donning preparation method and the donning method can be performed using the donning assistance tool 1 prepared in Example 1 (the diameter of the ring-forming member was 20 mm). Moreover, although not shown in the drawing, also in the case in which the donning assistance tool 1 prepared in Example 2 was used, it was confirmed that the donning preparation method and the donning method can be performed similarly to Example 1. In the case in which the donning assistance tool 1 prepared in Example 2 was used, since the risks of breakage of the connecting body were small when stretched, the donning preparation method and the donning method were easily performed. Moreover, the diameter returned to the non-stretched diameter in a short time after use. This is because the connecting body 3 with a restoring force stronger (the diameter of the connecting body 3 in a cross section is greater) than that of Example 1 was used, since it is unnecessary to tie the end portions of the connecting body 3 of Example 2. In the donning assistance tool 1 of Example 2, it is unnecessary to consider the connection of the end portions of the connecting body 3, and thus, it is possible to obtain the effect that the options of the connecting body 3 are widened.

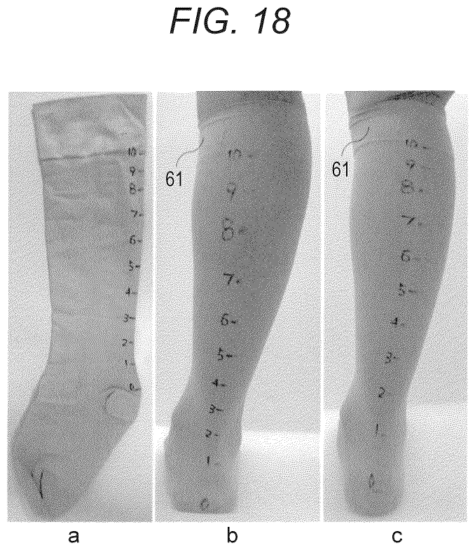

In a of FIG. 17a, photographs of the donning assistance tool 1, around which the end portion 61 of the elastic garment 6 was rolled, and in which the diameters of the ring-forming member are 30 mm and 10 mm, are shown. Even though the diameter of the ring-forming member 2 was 30 mm, the end portion 61 of the elastic garment 6 was able to roll up. However, the amount of the end portion 61 to be pulled off from the tubular product 5 was large, and the rolling up of the end portion 61 around the donning assistance tool 1 was slightly difficult. Moreover, even in the case in which the diameter of the ring-forming member 2 was 10 mm, the end portion 61 of the elastic garment 6 was able to roll up. However, since the diameter of the ring-forming member was small, the rolling operation and the rolling up operation were difficult. On the other hand, as shown in b of FIG. 17, in the case in which the donning assistance tool 1 with a diameter of the ring-forming member of 20 mm was used, the rolling operation of the end portion 61 of the elastic garment 6 and the rolling up operation of the elastic garment 6 were able to be easily performed. From the results described above, it was confirmed that preferably, the diameter of the ring-forming member ranges from 10 mm to 30 mm, and the rolling operation of the end portion 61 of the elastic garment 6 around the donning assistance tool 1 becomes difficult when the diameter is smaller than 10 mm or greater than 30 mm. Moreover, it was confirmed that the rolling up operation of the elastic garment 6 becomes difficult when the diameter of the ring-forming member 2 is smaller than 10 mm.

FIG. 18 shows photographs showing the difference in effects between the case of using the donning assistance tool 1 and the case of not using the donning assistance tool 1. More specifically, a of FIG. 18 is a photograph of a knee sock type elastic garment with scales, b of FIG. 18 is a photograph of an elastic garment donned with no use of the donning assistance tool 1, and c of FIG. 18 is a photograph of an elastic garment donned using the donning assistance tool 1. As shown in b of FIG. 18, in the case in which the elastic garment was donned with no use of the donning assistance tool 1, the site of the end portion of the elastic garment 61 was pulled, this caused the site near the end portion 61 to be stretched whereas caused the site near the toe to be not stretched, leading to unsuccessfully evenly donning the knee sock. On the other hand, as shown in c of FIG. 18, in the case in which the elastic garment was donned using the donning assistance tool 1, the elastic garment was able to be donned evenly.

### [Comparison to Commercially Available Products]

Next, it was confirmed that the difference between the case of using Magnide (see FIG. 1), Stocking Donner (see FIG. 2), and Doff N′Donner (see FIG. 3), which are commercially available donning assistance tools, and the case of using the donning assistance tool 1 disclosed in the present application. Table 1 shows the size and the traction force when used. The sizes of the commercially available products are the length of the longest part of the products shown in FIG. 1 to FIG. 3. Moreover, the size of the donning assistance tool 1 disclosed in the present application is the outer diameter of the ring body. The traction force when being used, shown in Table 1, is the values of traction force necessary when using the donning assistance tools measured using a soft compression knee sock type (Ansilk-1, made by ALCARE Co., Ltd.) as the elastic garment and a manual muscle meter (μTas F-1 made by ANIMA Corporation), (force necessary for operation shown by arrows in e of FIG. 1, e of FIG. 2, e of FIG. 3, and e of FIG. 14e). For comparison, the traction force in the case of using no donning assistance tool is also shown.

As is apparent from Table 1, in the donning assistance tool 1 disclosed in the present application, the size of the parts necessary for carrying was able to be significantly made smaller than the donning assistance tools of the commercially available products. Moreover, the traction force of the soft compression knee sock type (Ansilk-1, made by ALCARE Co., Ltd.) when donning was able to be made smaller than that of the commercially available products.

TABLE 1

| | Donning Assistance | | Stocking | Doff | No Donning |
|---|---|---|---|---|---|
| | Tool 1 | Magnide | Donner | N′Donner | Assistance Tool |
| Size | 12 cm | 25 cm | 36 cm | 56 cm | |
| Traction force | 3.0 | 5.7 | 9.0 | 3.2 | 7.8 |
| when used (kgf) | | | | | |

Most users of the donning assistance tool 1 are elderly people or individuals with conditions such as lower limb edema. When the size of the donning assistance tool 1 is portable, it is desirable for those people to don the elastic garment with a small force. According to the donning assistance tool 1 disclosed in the present application, it was confirmed that the different effects are obtained and exerted: a reduction in the size of the elastic garment and a reduction of the traction force when donning.

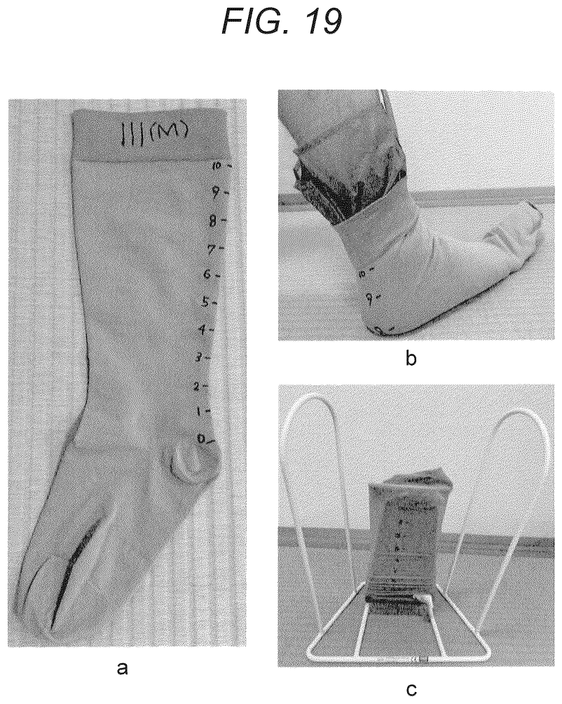

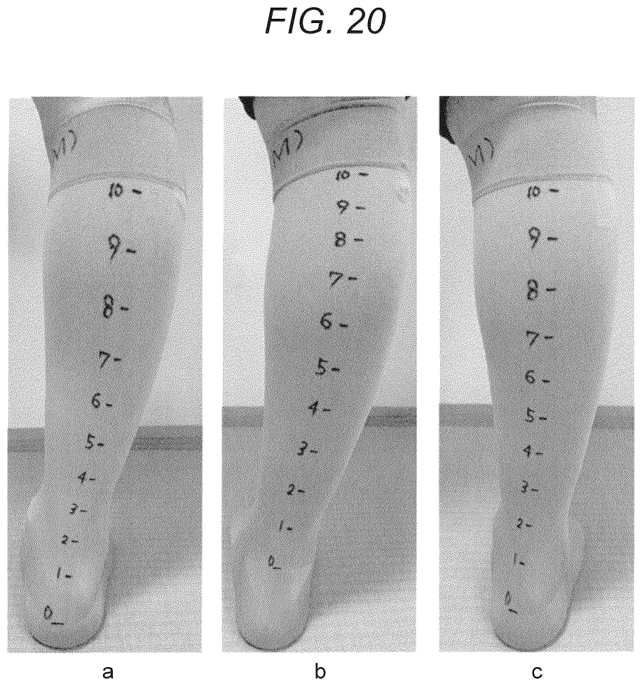

Next, experiments were conducted to confirm the difference in the donning situations in donning a knee sock using Magnide (see FIG. 1, the pull-out type), Stocking Donner (see FIG. 2, the pull-up type), which are the commercially available donning assistance tools, and the donning assistance tool 1 disclosed in the present application. Moreover, for the donning assistance tool 1, one prepared in Example 2 was used. In FIG. 19, a of FIG. 19 is a photograph of a knee sock-type elastic garment with scales, b of FIG. 19 is a photograph showing the preparation of donning using Magnide, and c of FIG. 19 is a photograph showing the preparation of donning using Stocking Donner. Moreover, the photographs of the experiment using the donning assistance tool 1 of Example 2 are omitted, since the photographs are the same as [Performing the donning preparation method and the donning method].

FIG. 20 shows photographs of donned knee socks. In FIG. 20, a of FIG. 20 is a photograph of a knee sock donned using Magnide (see FIG. 1, the pull-out type), b of FIG. 20 is a photograph of a knee sock donned using Stocking Donner (see FIG. 2, the pull-up type), and c of FIG. 20 is a photograph of a knee sock donned using the donning assistance tool 1 of Example 2. As shown in a of FIG. 20, in the pull-out type, the upper part of the knee sock was stretched and the lower part was shrunk. As shown in b of FIG. 20, in the pull-up type, the upper part of the knee sock was shrunk and the lower part was stretched. On the other hand, in the case of using the donning assistance tool 1 of Example 2, as shown in c of FIG. 20, the knee sock was evenly stretched. From the results as described above, in the donning assistance tool 1 disclosed in the present application, it was confirmed that the elastic garment can be evenly donned as compared to the commercially available products.

### INDUSTRIAL APPLICABILITY

It is possible to reduce the size of the donning assistance tool 1 disclosed in the present application compared to the previously existing donning assistance tools, and it is possible to don the elastic garment with a small force. Therefore, this is useful for the medical appliance industry, the nursing care industry, and the like.

### DESCRIPTION OF REFERENCE SIGNS

1 . . . donning assistance tool, ring body,
11 . . . virtual circle,
12 . . . diameter,
2 . . . ring-forming member,
21 . . . size of ring-forming member,
22 . . . through hole,
3 . . . connecting body,
4 . . . friction adjustment sheet,
41 . . . base material,
42 . . . fiber,
5 . . . tubular product,
51 . . . first end portion side,
52 . . . second end portion side,
6 . . . elastic garment,
61 . . . end portion of elastic garment,
7 . . . cup

1. A donning assistance tool for an elastic garment, the donning assistance tool comprising:
a plurality of ring-forming members; and
a connecting body that is formed of a stretchable and contractible material and that connects the ring-forming members, wherein
the ring-forming member has
a size ranging from 10 mm to 30 mm, and
a through hole into which the connecting body is inserted, and
regarding a size in a non-stretched state of a ring body formed by inserting the connecting body into the through hole, a diameter in placing the ring body such that a virtual line inscribed in the ring-forming members constituting the ring body ranges from 40 mm to 130 mm.

2. The donning assistance tool according to claim 1, wherein the ring-forming member has a cut portion reaching from the through hole to an outer edge of the ring-forming member, and
the connecting body is insertable into the through hole via the cut portion.

3. The donning assistance tool according to claim 2, wherein an inner diameter of the ring body is adjustable by adding the ring-forming member to the connecting body or removing the ring-forming member from the connecting body.

4. The donning assistance tool according to claim 1, wherein the through hole is formed at a position off-centered from approximately a center of the ring-forming member.

5. The donning assistance tool according to claim 1, wherein the ring-forming member is formed in a spherical shape.

6. The donning assistance tool according to claim 1, wherein a size of the through hole of the ring-forming member is almost equal to a size of an outer shape of the connecting body.

7. A donning assistance kit for an elastic garment, the donning assistance kit comprising:
the donning assistance tool according to claim 1; and
a friction adjustment sheet, wherein
friction of the friction adjustment sheet moved in a state in which the elastic garment is contacted is varied between a first direction and a second direction that is a direction opposite to the first direction.

8. A donning assistance kit for an elastic garment, the donning assistance kit comprising:
the donning assistance tool according to claim 1; and
a cup having an outer diameter of 69 mm or an outer diameter of 90 mm.

9-10. (canceled)

11. A donning preparation method for an elastic garment, wherein the donning preparation method is performed at least using
the donning assistance tool according to claim 1, and
a tubular product, and
the donning preparation method includes
an elastic garment end portion attaching process in which an end portion of the elastic garment is attached to the tubular product while sliding from a first end portion side to a second end portion side of the tubular product,
an elastic garment rolling up process in which the donning assistance tool is placed on the end portion of the elastic garment attached to the tubular product, the donning assistance tool is moved while rotating from the second end portion side to the first end portion side of the tubular product along the tubular product, and the elastic garment is rolled up around the donning assistance tool,
an elastic garment re-attaching process in which the donning assistance tool around which the elastic garment is rolled up is slid from the first end portion side to the second end portion side, and the elastic garment is again attached to the tubular product, and
an attached elastic garment re-rolling up process in which the donning assistance tool is moved while rotating from the second end portion side to the first end portion side of the tubular product along the tubular product, and the elastic garment again attached to the donning assistance tool is rolled up.

12-14. (canceled)

15. A donning method for an elastic garment comprising:
a donning process in which the elastic garment rolled up around the donning assistance tool by the donning preparation method according to claim 11 is donned on a hand or a foot.

16. The donning assistance tool according to claim 1, comprising the ring-forming member having a different size.

17. The donning assistance tool according to claim 1, wherein a part of the ring-forming members or an entire part of the ring-forming members is not rotatable with respect to the connecting body.
